(19) United States
(12) Patent Application Publication
(10) Pub. No.: US 2008/0021076 A1
(43) Pub. Date: Jan. 24, 2008

(54) Treatment of Pain

(76) Inventors: Ronald Rosenburg, Ingelheim (DE); Jeffrey Miller, Fairfield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/780,102

(22) Filed: Jul. 19, 2007

Related U.S. Application Data
(60) Provisional application No. 60/831,982, filed on Jul. 19, 2006.

(51) Int. Cl. A61K 31/428; A61P 25/04

(52) U.S. Cl. 514/367

(57) ABSTRACT

The invention relates to a new medicamentation for the treatment of pain, in particular fibromyalgia. The medicamentation comprises the administration of pramipexole and duloxetine. The medicamentation is a combination of pramipexole an duloxetine which may be used in a fixed dose combination as well as in a free dose combination. The invention further is related to the manufacture of a medicament for the treatment of pain, in particular fibromyalgia comprising said medicamentation and a method of treatment of pain, in particular fibromyalgia comprising said medicamentation.

The invention relates to a new medicamentation for the treatment of pain, in particular fibromyalgia. The medicamentation comprises the administration of pramipexole and duloxetine. The medicamentation is a combination of pramipexole and duloxetine which may be used in a fixed dose combination as well as in a free dose combination. The invention further is related to the manufacture of a medicament for the treatment of pain, in particular fibromyalgia comprising said medicamentation and a method of treatment of pain, in particular fibromyalgia comprising said medicamentation.

In the context of the present invention pain shall be used as a collective term for several complex forms of sensory perception, characterised by the disturbance of feeling well. Usually, one perceives pain in its acute form. However, pain can develop into a chronic form, which itself is considered to be a discrete disease. Pain is divided into at least three subfamilies: a) nociceptive pain with excitation of the pain receptors and transmission of the impact to the CNS; b.) neuropathic pain as consequence of tissue damages and/or damages and/or injuries of the peripheric or central nervous system, in particular in the form of diabetic polyneuropathy; c.) pain following functional dysfunction, e.g. migraine, back pain or psychosomatic processes.

In the context of the present invention the most prominent forms of pain are neuropathic pain, head ache, in particular migraine and/or fibromyalgia. Fibromyalgia is the most preferred form of an illness associated with the present invention.

### BACKGROUND

Neuropathic pain or painful peripheral neuropathy can be classified by the type of nerve that has been injured or damaged. Basically, one distinguishes between three types of nerves, motor nerves, sensory nerves and autonomic nerves. Another way of describing neuropathic pain is by referring to the area that is affected. If only one area is affected the disease is called mononeuropathy, if several areas are affected, the disease is called polyneuropathy. There are many causes under discussion that can lead to neuropathy, e.g. diseases like diabetes, autoimmune disorders, Bell's palsy, cancer, Charcot-Marie-Tooth disease, Carpal tunnel syndrome, chronic kidney failure, connective tissue disease, liver failures; intoxication; nutritional causes like alcoholism, vitamin deficiencies and so on. Migraine is an intense and disabling episodic form of headache. The pain of a migraine headache is often described as an intense pulsing or throbbing pain, predominantly in one area of the head. It is often accompanied by extreme sensitivity to light and sound, nausea, and vomiting. Some Warning signals for an episode may be a so called “aura,” visual disturbances that appear as flashing lights or a temporary loss of vision. People with migraine tend to have recurring attacks triggered by a lack of food or sleep, exposure to light, or hormonal irregularities (only in women). Anxiety, stress, or relaxation after stress can also be triggers.

Fibromyalgia is a chronic disorder characterized by widespread musculoskeletal pain and tenderness to palpation at specific tender points. In addition fibromyalgia patients often have other symptoms such as fatigue, sleep disturbances, headache or cognitive dysfunction. The American College of Rheumatology has defined Fibromyalgia as pain in all four quadrants and axial skeletal pain, along with at least 11 of 18 tender point sites. Widespread pain must have been present for at least 3 months. Tender points, the diagnostic hallmark of fibromyalgia, are examples of hyperalgesia, thought to be due to central sensitization. Patients with fibromyalgia have quantitatively altered nociception compared to pain-free patients, suggesting that people with fibromyalgia process sensory information differently, most likely due to changes in the central processing of pain at the spinal level.

Patients may have widespread pain over all parts of the body which often seems to arise in the muscles. The most common sites of pain include the neck, back, shoulders, pelvic girdle and hands, but any body part can be involved. The pain shows varying intensities that wax and wane over time, it is profound, widespread and chronic. The pain is described as deep muscular aching, throbbing, twitching, stabbing and shooting pain. Neurological complaints such as numbness, tingling and burning are often present. The severity of the pain and stiffness is often worse in the morning. Aggravating factors that affect pain include cold/humid weather, non-restorative sleep, physical and mental fatigue, excessive physical activity, physical inactivity, anxiety and stress. Additionally to pain, patients commonly complain of fatigue in form of an all-encompassing exhaustion that interferes with even the simplest daily activities. Within the spectrum of symptoms are a decreased sense of energy, disturbances of sleep, problems with memory and concentration and varying degrees of anxiety and depression. Furthermore, certain other medical conditions are sometimes associated with fibromyalgia, such as: tension headaches, migraine, irritable bowel syndrome, overactive bladder, pelvic pain, premenstrual tension syndrome, cold intolerance, dry eyes and mouth, anxiety, depression, ringing in the ears, dizziness, vision problems and others. Patients with established rheumatoid arthritis, lupus (SLE) and Sjogren's syndrome often develop fibromyalgia symptoms during the course of their disease.

The use of duloxetine, N-methyl-3-(1-naphthalenyloxy)-3-(2-thienyl)propanamine and any pharmaceutically acceptable salts thereof (hereinafter referred to as “duloxetine”) in the manufacture of a medication to treat fibromaylgia is known from U.S. Pat. No. 6,596,756.

The use of pramipexole, 2-amino-6-n-propylamino-4,5,6,7-tetrahydrobenzo-thiazole, preferably the (−)-enantiomers as well as any pharmaceutically acceptable salts of any of them (hereinafter referred to as pramipexole) in the manufacture of a medication to treat fibromyalgia is known in the art.

### SUMMARY OF THE INVENTION

One objective of the present invention is the use of pramipexole in the combination with duloxetine for the manufacture of a medicament for the treatment of pain, which preferably is fibromyalgia or neuropathic pain or headache or migraine. Preferred is the treatment of fibromyalgia. The use may be either a free-dose combination or a fixed-dose combination.

### DESCRIPTION OF THE INVENTION

The present invention is based on the concept of a combined application of pramipexole and duloxetine in order to treat the aforementioned kinds of pain.

The effective amount or dose of duloxetine for treating pain is in the range from about 1 mg/day to about 200 mg/day. The preferred adult dose is in the range from about 40 to about 150 mg/day, and a more highly preferred adult dose is from about 60 to about 120 mg/day. The optimum dose for each patient, as always, must be set by the physician in charge of the case, taking into account the patient's size, other medications which the patient requires, severity of the persistent pain and all of the other circumstances of the patient.

Since duloxetine is readily orally absorbed and requires only once/day administration, there is little or no reason to administer it in any other way than orally. It is produced in the form of a clean, stable crystal, and thus is easily formulated in the usual oral pharmaceutical forms, such as tablets, capsules, suspensions, and the like. The usual methods of pharmaceutical scientists are applicable. It may usefully be administered, if there is any reason to do so in a particular circumstance, in other pharmaceutical forms, such as, but not limited to, injectable solutions, depot injections, suppositories and the like, which are well known to and understood by pharmaceutical scientists. It will substantially always be preferred, however, to administer duloxetine as a tablet or capsule and such pharmaceutical forms are recommended.

A preferred duloxetine enteric formulation as disclosed in U.S. Pat. No. 5,508,074, which is hereby incorporated by reference, is a pellet formulation comprising a) a core consisting of duloxetine and a pharmaceutically acceptable excipient; b) an optional separating layer; c) an enteric layer comprising hydroxypropylmethylcellulose acetate succinate (HPMCAS) and a pharmaceutically acceptable excipient; d) an optional finishing layer. The following example demonstrates the preparation of a preferred such formulation.

The effective amount or dose of pramipexole, in particular in form of the dihydrochloride monohydrate for treating pain is in the range from about 0.1 mg/day to about 10 mg/day. The preferred adult dose is in the range from about 0.2 to about 6 mg/day, and a more highly preferred adult dose is from about 0.4 to about 5 mg/day. The optimum dose for each patient must be set by the physician in charge of the case, taking into account the patient's size, other medications which the patient requires, severity of the persistent pain and all of the other circumstances of the patient.

In the treatment of pain, in particular a chronic kind of pain, it may be recommendable to apply pramipexole in an extended release form, a suitable one of which is disclosed in WO 2006/015942 or WO 2006/015943, both of which are hereby incorporated by reference.

An extended release tablet according to WO 2006/015942 and applicable in the context of the invention is characterised in that the extended release formulation comprises pramipexole or a pharmaceutically acceptable salt thereof in a matrix comprising at least one water swelling polymer, preferably other than pregelatinized starch. The matrix preferably comprises at least two water swelling polymers preferably other than pregelatinized starch, and wherein at least one of the at least two polymers is an anionic polymer. The anionic polymer preferably is selected from the group of optionally crosslinked acrylic acid polymers, methacrylic acid polymers, alginates and carboxymethylcellulose. The anionic polymer is an optionally crosslinked acrylic acid polymer, wherein the content of the optionally crosslinked acrylic acid polymer in the matrix is from about 0.25 wt.-% to about 25 wt.-%, and preferably from about 0.5 wt.-% to about 15 wt.-%, and preferably from about 1 wt.-% to about 10 wt.-%. Optionally, at least one of the at least two polymers is a substantially neutral polymer, preferably other than pregelatinized starch. Preferably, the substantially neutral polymer is selected from hydroxypropyl cellulose and hydroxypropylmethyl cellulose. More preferably the substantially neutral polymer is hydroxypropyl methylcellulose, and wherein the content of hydroxypropyl methylcellulose in the matrix is from about 10 wt.-% to about 75 wt.-%, and preferably from about 25 wt.-% to about 65 wt.-%.

In one embodiment the matrix comprises about:

| | | |
|---|---|---|
| (a) pramipexole or a salt thereof | 0.05 to 5 | wt.-% |
| (b) anionic water swelling polymer(s) | 0.25 to 25 | wt.-% |
| (c) neutral water swelling polymer(s) | 10 to 75 | wt.-% |
| (d) further excipients | ad 100 | wt.-% |

In one embodiment the matrix comprises
(a) at least one water swelling polymer other than pregelatinized starch and optionally excipients, the resulting tablet providing a pH-independent in vitro release characteristic in the range from pH 1 to 7.5, or
(b) at least one water swelling anionic polymer and optionally excipients, the resulting tablet providing a pH-dependent release characteristic with a preferably faster release characteristic in the range of pH<4.5, and a slower and further on pH-independent release characteristic in the range from pH 4.5 to 7.5.

Such an extended release tablet may have a non-functional coating.

Preferably, such tablet is for a once daily application.

An extended release pellet formulation according to WO 2006/015943 and applicable in the context of the invention is characterised in that it comprises an active ingredient selected from pramipexole and the pharmaceutically acceptable salts thereof, and at least one release-modifying excipient. Preferably, the active ingredient is embedded within a matrix formed by the at least one release-modifying excipient, which is preferably selected from the group of lipids, waxes, and water-insoluble polymers. Preferably, it comprises a core and a coating, wherein at least one release-modifying excipient is incorporated in the coating and optionally the active ingredient is incorporated in the core. Such a coating may comprise at least a first layer and a second layer surrounding the first layer, wherein the first layer comprises the active ingredient, and wherein the second layer comprises at least one release-modifying excipient, preferably selected from ethylcellulose, cellulose acetate, polyvinylacetate, polyacrylates, polymethacrylates, and ammonio methacrylate copolymer. The second layer further may comprise at least one water-soluble excipient, preferably selected from hydroxypropylcellulose, hydroxypropyl methylcellulose, polyvinylpyrrolidone and polyethylene glycol. The second layer may further comprise an enteric coating polymer, preferably selected from methacrylic acid copolymers type A and B.

In one embodiment, the second layer comprises from about 10 to about 85 wt.-% of the enteric coating polymer and from about 15 to about 75 wt.-% of the water-insoluble polymer.

The core may comprise a saccharide, such as saccharose, starch, cellulose and a cellulose derivative, preferably microcrystalline cellulose.

In one embodiment, the extended release pellet formulation comprises
an inert pellet core;
a first layer being an active ingredient layer comprising pramipexole or a pharmaceutically acceptable salt thereof and optionally one or more wet binders and further excipients; and
a second layer provided on the first layer, the second layer being an extended release coating comprising
   (a) at least one water-insoluble polymer and optionally a pore former, the resulting pellet having a pH-independent in vitro release characteristic or
   (b) a mixture of a pH-dependent enteric-coating polymer and a pH-indenpendently water swelling polymer, the resulting pellet having a close to zero order in vitro release characteristic at acidic pH values up to pH 6.8, an accelerated release above pH 6.8 and a more accelerated release above pH 7.3.

The inert pellet core may comprise polysaccharides, cellulose, a cellulose derivative, starch and/or waxes. The inert pellet core further may comprise saccharose and/or microcrystalline cellulose, preferably microcrystalline cellulose.

Such an extended release pellet formulation using active pellets containing pramipexole may be prepared by wet or melt extrusion or melt granulation instead of pellets prepared by drug substance layering onto inert pellet cores.

The water-insoluble polymer of the extended release pellets may be selected from the group consisting of ethylcellulose, cellulose acetate, polyvinylacetate, polyacrylates and derivatives, such as quarternary ammonium substituted acrylic polymer, preferably ammonio methacrylate copolymer, type B, and ethylcellulose, most preferably ethylcellulose.

The pH-dependent enteric-coating polymer may be an anionic carboxylic acrylic polymer, preferably a partly methyl esterified methacrylic acid polymer, soluble above a pH value of 5.5, preferably above a pH value of 7.0.

The pH-independently water swelling polymer also may be a quarternary ammonium substituted acrylic polymer, preferably having an ammonium substitution of about 5 to about 10 per cent by weight.

The pH-dependent enteric-coating polymer may be present in an amount of 10 to 85% by weight of the coating and the pH-independently water swelling polymer is present in an amount of 15 to 75% by weight of the coating.

The extended release coating may additionally contain a pore-forming component.

The pore-forming component may be selected from the group consisting of hydroxypropylcellulose, hydroxypropyl methylcellulose, polyvinylpyrrolidone and polyethylen glycol, preferably selected hydroxypropylcellulose from the Klucel series.

The extended release pellet formulation containing an active ingredient selected from pramipexole and the pharmaceutically acceptable salts thereof may be prepared by wet or melt extrusion or melt granulation using excipients achieving extended release without a further diffusion membrane.

The pellets may be applied in form of a capsule, which comprises a sufficient number of pellets to provide a daily dose administered at one time.

To the extent that any pharmaceutically active compound is disclosed or claimed, it is expressly intended that all active metabolites which are produced in vivo are included, and it is expressly intended that all enantiomers, diastereomers or tautomers are included, if the compound is capable of occurring in its enantiomeric, diastereomeric or tautomeric form. Obviously, the isomer which is pharmacologically most effective and most free from side effects is preferred.

Both compounds can be administered in form of a pharmaceutically acceptable salt. Examples of pharmaceutically active salts for each of the compounds which are the subject of this description include, without being restricted thereto, salts which are prepared from pharmaceutically acceptable acids or bases, including organic and inorganic acids and bases. As duloxetine and pramipexole both are basic as neutral compounds, salts may be prepared from pharmaceutically acceptable acids. When selecting the most preferred salt, or to clarify whether a salt or the neutral compound is used, properties such as bioavailability, ease of manufacture, workability and shelf life are taken into consideration, inter alia. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like. Examples of pharmaceutically acceptable salts include, without being restricted thereto, acetate, benzoate, hydroxybutyrate, bisulphate, bisulphite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulphonate, methoxybenzoate, methylbenzoate, monohydrogenphosphate, naphthalene-1-sulphonate, naphthalene-2-sulphonate, oxalate, phenylbutyrate, phenylproprionate, phosphate, phthalate, phenylacetate, propanesulphonate, propiolate, propionate, pyrophosphate, pyrosulphate, sebacate, suberate, succinate, sulphate, sulphite, sulphonate, tartrate, xylenesulphonate and the like.

The two active compounds, duloxetine and pramipexole may be subject to one single pharmaceutical formulation or they may be applied as discrete separate pharmaceutical formulations. The advantage of the first variant is that the doses are fixed in this pharmaceutical formulation. In such a case the pharmaceutical formulation is called a “fixed-dose-combination”. The advantage of the second variation is that each compound can be applied in free eligible dosage form. Such a “free-dose combination” allows for to better titrate a patient if the dosage of one of the two components of the combination therapy should be lowered or raised in relation to the other one in order to increase efficacy. In case of free-dose combinations, the two application forms, (pramipexole application form and duloxetine combination form), may be applied together, within a short period of time (within 60 minutes, more preferably 30 minutes, more preferably 10 minutes) or within a long period of time (within 24 hours, more preferably 12 hours, more preferably 6 hours and more preferably 1 hour). Preferably the two kinds of drugs are taken within 5 minutes.

In the case of fixed dose combination in form of an extended release formulation, the same may be prepared on basis of the aforementioned pramipexole comprising extended release formulations, in particular the ones according to WO 2006/015942 or WO 2006/015943, the characteristics of which have been outlined above, duloxetine may be added to the same in the appropriate dosage as outlined in this description.

In case of fixed dose combination in form of an immediate release formulation, the same may be prepared on basis of the immediate formulations as outlined in this description for each of the two combination partners.

Other formulations on basis of the herein described formulations comprising only one of the combination partners may be prepared as well.

In the following the invention shall be illustrated in form of formulations which may be freely combined:

### EXAMPLES

Formulations Concerning Pramipexole

a.) Immediate Release Formulations:
Tablet comprising 0.125 mg pramipexole-dihydochloride-monohydrate or 0.25 mg thereof or 0.5 mg thereof, or 1 mg thereof in combination with mannitol, corn starch, highly disperse silicium dioxide, povidon, magnesium stearate. This formulation is known in the market as Sifrol® or Mirapex® (immediate release formulation).

b.) Extended Release Formulations:

ba. pramipexole extended release tablets

| | |
|---|---|
| | mg per 0.75 |
| Ingredient | mg tablet |

ba.1

| | |
|---|---|
| Pramipexole-dihydrochloride monohydrate, peg-milled | 0.750 |
| Hypromellose 2208 | 157.500 |
| (Methocel K 15 M) | |
| Corn starch | 183.700 |
| Carbomer 941 | 3.500 |
| (Carbopol ® 71 G) | |
| Colloidal Silicon dioxide | 2.800 |
| Magnesium stearate | 1.750 |
| Total | 350.000 |

ba.2

| Pramipexole-dihydrochloride monohydrate, peg-milled | 0.750 |
|---|---|
| Hypromellose 2208 | 157.500 |
| (Methocel K 15 M) | |
| Corn starch | 174.600 |
| Carbomer 941 | 14.000 |
| (Carbopol ® 71 G) | |
| Colloidal Silicon dioxide | 1.400 |
| Magnesium stearate | 1.750 |
| Total | 350.000 |
| Constituents | mg/tablet |

ba.3

| | |
|---|---|
| Pramipexole-dihydrochloride monohydrate, peg-milled | 0.750 |
| Hypromellose 2208 | 157.500 |
| (Methocel K 100 M) | |
| Corn starch | 187.900 |
| Colloidal silicon dioxide | 2.100 |
| Magnesium stearate | 1.750 |
| Total weight matrix tablet | 350.000 |

ba.4

| | |
|---|---|
| Pramipexole-dihydrochloride monohydrate, peg-milled | 0.750 |
| Hypromellose 2208 | 175.000 |
| (Methocel K 15 M) | |
| Carboxymethylcellulose sodium | 87.500 |
| Lactose monohydrate (200 mesh) | 52.500 |
| Microcrystalline cellulose (grade PH 101) | 31.100 |
| Colloidal silicon dioxide | 1.400 |
| Magnesium stearate | 1.750 |
| Total weight matrix tablet | 350.000 |

ba.5

| | |
|---|---|
| Pramipexole-dihydrochloride monohydrate, peg-milled | 0.750 |
| Hypromellose 2208 | 175.000 |
| (Methocel K 15 M) | |
| Carboxymethylcellulose sodium | 87.500 |
| Lactose monohydrate (200 mesh) | 52.500 |
| Microcrystalline cellulose (grade PH 101) | 27.600 |
| Carbomer 941 (Carbopol ® 71 G) | 3.500 |
| Colloidal silicon dioxide | 1.400 |
| Magnesium stearate | 1.750 |
| Total weight matrix tablet | 350.000 |

ba.6

| | |
|---|---|
| Pramipexole-dihydrochloride monohydrate, peg-milled | 0.750 |
| Hypromellose 2208 | 175.000 |
| (Methocel K 15 M) | |
| Carboxymethylcellulose sodium | 87.500 |
| Lactose monohydrate (200 mesh) | 45.500 |
| Microcrystalline cellulose (grade PH 101) | 24.100 |
| Carbomer 941 (Carbopol ® 71 G) | 14.000 |
| Colloidal silicon dioxide | 1.400 |
| Magnesium stearate | 1.750 |
| Total weight matrix tablet | 350.000 |

ba.7

| | |
|---|---|
| Pramipexole-dihydrochloride monohydrate, peg-milled | 0.750 |
| Carbomer 941 (Carbopol ® 71 G) | 87.500 |
| Lactose monohydrate (200 mesh) | 225.400 |
| Microcrystalline cellulose (grade PH 101) | 33.200 |
| Colloidal silicon dioxide | 1.400 |
| Magnesium stearate | 1.750 |
| Total weight matrix tablet | 350.000 |

ba.8

| | |
|---|---|
| Pramipexole-dihydrochloride monohydrate, peg-milled | 0.750 |
| Carbomer 941 (Carbopol ® 71 G) | 70.000 |
| Lactose monohydrate (200 mesh) | 242.900 |
| Microcrystalline cellulose (grade PH 101) | 33.200 |
| Colloidal silicon dioxide | 1.400 |
| Magnesium stearate | 1.750 |
| Total weight matrix tablet | 350.000 |

ba.9

| | |
|---|---|
| Pramipexole-dihydrochloride monohydrate, peg-milled | 0.750 |
| Carbomer 941 (Carbopol ® 71 G) | 70.000 |
| Lactose monohydrate (200 mesh) | 140.000 |
| Calcium Phosphate, dibasic dihydrate | 136.100 |
| Colloidal silicon dioxide | 1.400 |
| Magnesium stearate | 1.750 |
| Total weight matrix tablet | 350.000 |

ba.10

| | |
|---|---|
| Pramipexole-dihydrochloride monohydrate, peg-milled | 0.750 |
| Carbomer 941 (Carbopol ® 71 G) | 52.500 |
| Lactose monohydrate (200 mesh) | 140.000 |
| Calcium Phosphate, dibasic dihydrate | 153.600 |
| Colloidal silicon dioxide | 1.400 |
| Magnesium stearate | 1.750 |
| Total weight matrix tablet | 350.000 |

ba.11

| | |
|---|---|
| Pramipexole-dihydrochloride monohydrate, peg-milled | 0.750 |
| Hypromellose 2208 | 157.500 |
| (Methocel K 15 M) | |
| Corn starch | 163.400 |
| Carbomer 941 (Carbopol ® 71 G) | 24.500 |
| Colloidal silicon dioxide | 2.100 |
| Magnesium stearate | 1.750 |
| Total weight matrix tablet | 350.000 |

ba.12

| | |
|---|---|
| Pramipexole-dihydrochloride monohydrate, peg-milled | 0.750 |
| Hypromellose 2910 | 0.788 |
| (Methocel E 5) | |
| Corn starch | 173.812 |
| Hypromellose 2208 | 157.500 |
| (Methocel K 15 M) | |
| Carbomer 941 (Carbopol ® 71 G) | 14.000 |
| Colloidal silicon dioxide | 1.400 |
| Magnesium stearate | 1.750 |
| Total weight matrix tablet | 350.000 |

ba.13

| | |
|---|---|
| Pramipexole-dihydrochloride monohydrate, peg-milled | 0.750 |
| Hypromellose 2208 | 148.500 |
| (Methocel K 15 M) | |
| Corn starch | 160.620 |
| Carbomer 941 (Carbopol ® 71 G) | 16.500 |
| Colloidal silicon dioxide | 1.980 |
| Magnesium stearate | 1.650 |
| Total weight matrix tablet | 330.000 |

bb. pramipexole extended release capsule

| | | |
|---|---|---|
| | mg per | mg per |
| | 0.75 mg | 0.75 mg |
| Ingredient | capsule | capsule |

bb.1

| | | |
|---|---|---|
| ER Pellets | 88.458 | |
| consisting of: | | |
| Pramipexole-dihydrochloride | | 0.750 |
| monohydrate | | |
| Microcrystalline cellulose | | 73.980 |
| pellets | | |
| (Cellets 700) | | |
| Hydroxypropyl Cellulose | | 0.150 |
| (Klucel EF) | | |
| Talc | | 0.495 |
| Methacrylic Acid Copolymer, | | 7.500 |
| Type B | | |
| (Eudragit S 100) | | |
| Ammonio Methacrylate | | 3.750 |
| Copolymer, Type B | | |
| (Eudragit RS 100) | | |
| Triacetin | | 1.833 |
| Ethanol (96%) | | 173.333* |
| Purified water | | 30.000* |
| HPMC capsule, size 3 | 46.000 | |
| Total | 134.458 | 88.458 |

bb.2

| | | |
|---|---|---|
| ER Pellets | 91.600 | |
| consisting of: | | |
| Pramipexole-dihydrochloride | | 0.750 |
| monohydrate | | |
| Microcrystalline cellulose | | 73.980 |
| pellets | | |
| (Cellets 700) | | |
| Hydroxypropylcellulose | | 0.150 |
| (Klucel EF) | | |
| Talc | | 0.578 |
| Methacrylic Acid Copolymer, | | 9.250 |
| Type B | | |
| (Eudragit S 100) | | |
| Ammonio Methacrylate | | 4.625 |
| Copolymer, Type B | | |
| (Eudragit RS 100) | | |
| Triacetin | | 2.267 |
| Ethanol (96%) | | 214.167* |
| Purified water | | 30.000* |
| HPMC capsule, size 3 | 46.000 | |
| Total | 137.600 | 91.600 |

bb.3

| | | |
|---|---|---|
| ER Pellets | 80.063 | |
| consisting of: | | |
| Pramipexole-dihydrochloride | | 0.750 |
| monohydrate | | |
| Microcrystalline cellulose | | 73.980 |
| pellets | | |
| (Cellets 700) | | |
| Hydroxypropylcellulose | | 0.150 |
| (Klucel EF) | | |
| Talc | | 0.495 |
| Ethylcellulose | | 3.750 |
| (N14) | | |
| Macrogol 6000 | | 0.938 |
| Ethanol (96%) | | 49.167* |
| Purified water | | 32.583* |
| HPMC capsule, size 3 | 46.000 | |
| Total | 126.063 | 80.063 |

bb.4

| | | |
|---|---|---|
| ER Pellets | 82.088 | |
| consisting of: | | |
| Pramipexole-dihydrochloride | | 0.750 |
| monohydrate | | |
| Microcrystalline cellulose | | 73.980 |
| pellets | | |
| (Cellets 700) | | |
| Hydroxypropylcellulose | | 0.150 |
| (Klucel EF) | | |
| Talc | | 0.645 |
| Ethylcellulose | | 5.250 |
| (N14) | | |
| Macrogol 6000 | | 1.313 |
| Ethanol (96%) | | 68.333* |
| Purified water | | 33.667* |
| HPMC capsule, size 3 | 46.000 | |
| Total | 128.088 | 82.088 |

bb.5

| | | |
|---|---|---|
| ER Pellets | 93.668 | |
| consisting of: | | |
| Pramipexole-dihydrochloride | | 0.750 |
| monohydrate | | |
| Microcrystalline cellulose | | 73.980 |
| pellets | | |
| (Cellets 700) | | |
| Hydroxypropylcellulose | | 0.630 |
| (Klucel EF) | | |
| Talc | | 1.995 |
| Methacrylic Acid Copolymer, | | 9.000 |
| Type B | | |
| (Eudragit S 100) | | |
| Ammonio Methacrylate | | 4.500 |
| Copolymer, Type B | | |
| (Eudragit RS 100) | | |
| Triethylcitrate | | 2.813 |
| Ethanol (96%) | | 250.200* |
| Purified water | | 30.000* |
| HPMC capsule, size 3 | 46.000 | |
| Total | 139.668 | 93.668 |

*removed during processing, does not appear in the final product

bb.8

Pellets Prepared by Melt Extrusion

In order to achieve adequate content uniformity, 9 g stearyl alcohol is mixed with 1 g of pramipexole. Then this mixture is mixed with 90 g stearyl alcohol. The mixture is extruded in a twin screw extruder at 51° C., diameter of dye is 0.7 mm using a face cut granulator to achieve pieces of about 1 mm. These are rounded in a spheronizer at 400 rpm and 41° C. The pellets are sieved, the fraction of 0.8-1.1 mm is used for retardation as described in the previous examples. Table 11 provides some further examples of melt extrusion.

Examples for Melt Extrusion:

| Example No | Pramipexole [g] | Stearyl alcohol [g] | Cetyl alcohol [g] |
|---|---|---|---|
| 8 | 1 | 99 | 0 |
| 8a | 0.5 | 59.5 | 40 |
| 8b | 2 | 58 | 40 |
| 8c | 0.5 | 49.5 | 50 |

bb.9

Extended Release Pellets Prepared by Wet Extrusion

In order to achieve adequate content uniformity, 9 g microcrystalline cellulose is mixed with 1 g of pramipexole. Then this mixture is mixed with 60 g g microcrystalline cellulose and 30 g carbomer 971P. The mixture is extruded in a twin screw extruder with an adequate amount of water (or binder solution), diameter of dye is 0.7 mm. The resulting extrudates are rounded in a spheronizer at 400 rpm. After drying, pellets are sieved, the fraction of 0.8-1.1 mm is filled into capsules.

| | | Microcrystalline | Extended release |
|---|---|---|---|
| Example No | Pramipexole [g] | cellulose [g] | excipient [g] |
| 9 | 1 | 69 | 30 carbomer 971P |
| 9a | 0.5 | 69.5 | 30 carbomer 971P |
| 9b | 2 | 68 | 30 carbomer 971P |
| 9c | 1 | 69 | 30 Eudragit S |
| 9d | 2 | 58 | 40 Eudragit S |
| 9e | 1 | 44 | 30 Eudragit S |
| | | | 25 carbomer 971P |

bb.10

Extended Release Pellets Prepared by Melt Extrusion

In order to achieve adequate content uniformity, 9 g hydrogenated castor oil is mixed with 1 g of pramipexole. Then this mixture is mixed with 60 g hydrogenated castor oil and 30 g carnauba wax. The mixture is extruded in a twin screw extruder with an adequate amount of water (or binder solution), diameter of dye is 0.7 mm. The resulting extrudates are rounded in a spheronizer at 400 rpm. Pellets are sieved, the fraction of 0.8-1.1 mm is filled into capsules.

| | | hydrogenated castor | |
|---|---|---|---|
| Example No | Pramipexole [g] | oil [g] | carnauba wax [g] |
| 10 | 1 | 69 | 30 |
| 10a | 0.5 | 69.5 | 30 |
| 10b | 2 | 68 | 30 |
| 10c | 1 | 59 | 40 |
| 10d | 1 | 78 | 21 |
| 10e | 1 | 83 | 16 |

bb.11

Extended Release Pellets Prepared by Hot Melt Granulation/Melt Pelletization

In this process agglomeration of active ingredient with excipients is promoted by the addition of low melting point, lipophilic binders, such as waxes, fats, fatty acids, fatty acid alcohols, and more water soluble polymers, such as poloxamers or polyethylene glycols. The binder is usually added to the other components as a powder. The binder is liquefied by heat generated either by friction during the mixing phase or by a heating jacket. Excipients suitable are e.g. lactose, microcrystalline cellulose, and dibasic calcium phosphate. After melting and granulation of the mass, the resulting mass is either cooled down, screened and processed into tablets together with further excipients or, spheronized into pellets, which can be coated in addition, and filled into capsules

| Example | | | Stearyl alcohol | carnauba wax |
|---|---|---|---|---|
| No | Pramipexole [%] | Lactose | [%] | [%] |
| 11 | 0.9 | 74.1 | 15 | 10 |
| 11a | 1.4 | 58.6 | 15 | 25 |
| 11b | 0.9 | 79.1 | 15 | 5 |

Formulations Concerning Duloxetine

| Beads | mg |
|---|---|
| sucrose-starch nonpareils | |
| 20-25 mash | |
| duloxetine-layer | 11.21 |
| duloxetine | 11.21 |
| hydroxypropylmethylcellulose | 3.74 |
| separating layer | |
| hydroxypropylmethylcellulose | 2.51 |
| talc, 500 mesh | 10.03 |
| sucrose | 5.0 |
| enteric layer | |
| hydroxypropylmethylcellulose acetate | 25.05 |
| succinate | |
| triethylcitrat | 5.0 |
| talc, 500 mesh | 7.52 |
| finishing layer | |
| hydroxypropylmethylcellulose | 8.44 |
| titanium dioxide | 2.81 |
| talc | trace |

What is claimed is:

1. A method of treatment for pain comprising the administration to a host suffering from pain an effective amount of pramipexole and an effective amount of duloxetine.

2. The method of claim 1, wherein the pain is due to fibromyalgia.

3. The method of claim 1, wherein the pain is due to migraine.

4. The method of claim 1, wherein the pain is chronic pain.

5. The method of claim 1, wherein the pain is neuropathic pain.

6. The method of claim 1, wherein the pramipexole is within an extended release formulation.

7. A pharmaceutical composition comprising an effective amount of pramipexole and an effective amount of duloxetine.

8. The pharmaceutical composition of claim 9, wherein it is an immediate release formulation.

9. The pharmaceutical composition of claim 9, wherein it is an extended release formulation.
